(19) United States
(12) Patent Application Publication
(10) Pub. No.: US 2009/0208894 A1
(43) Pub. Date: Aug. 20, 2009

(54) Curing Light

(75) Inventors: Marc Orloff, Glendale, CA (US); John Murtagh, Los Angeles, CA (US); Travis Pham, Los Angeles, CA (US)

Correspondence Address: DISCUS DENTAL, LLC, 8550 HIGUERA STREET, CULVER CITY, CA, 90232, US

(73) Assignee: Discus Dental, LLC, Culver City, CA (US)

(21) Appl. No.: 12/388,493

(22) Filed: Feb. 18, 2009

Related U.S. Application Data
(60) Provisional application No. 61/029,558, filed on Feb. 18, 2008.

(51) Int. Cl. A61N 5/06

(52) U.S. Cl. 433/29

(57) ABSTRACT

The present invention relates to curing light devices for use in dentistry. Specifically, this invention relates to curing light devices generating convergent and uniform light with high intensity. The curing light device has a handle portion and a front portion wherein a light module is located in a desirable position in the interior of the front portion. The light module may include a light source comprising at least three LEDs in a compact manner, an optical element adapted to direct and/or focus the LEDs, and a lens cap to protect the LEDs and the optical elements. The lens cap may include a peripheral groove extending more than half of the periphery of the lens cap, which provides a longer engagement portion and thus a more secured attachment when “over-centeredly” engaging with a receiving edge on the light emitting end. A substantially precise alignment is achieved between the LEDs and corresponding lenses on the optical element to minimize heat dissipation and to increase the runtime and intensity of the curing light device. The curing light has an effective beam diameter of at least about 9 mm with output homogeneity of up to about 6 mm away from the emitting end.

### CROSS-REFERENCE TO RELATED APPLICATIONS

This application claims the benefit of U.S. provisional patent application Ser. No. 61/029,558, filed Feb. 18, 2008, entitled “Curing Light”, the contents of which are hereby incorporated by reference.

### FIELD OF THE INVENTION

This invention relates to a curing light device for use in dentistry. Specifically, this invention relates to a portable curing light device for dental curing purposes.

### BACKGROUND OF THE INVENTION

In the field of dentistry, tooth restoration and repaired, dental cavities are often filled and/or sealed with compounds that are photosensitive, either to visible and/or ultraviolet light. These compounds, commonly known as light-curable compounds, are placed within dental cavity preparations or onto dental surfaces and are cured when exposed to light from a dental curing light device.

Composite resin fillings have become the standard for filling cavities in dentistry today. These composites fillings use resins that must be cured after application. Hand-held curing lights have been extensively used for this curing purpose. Some of them are constructed with fiber optic light wands designed for directing light from the light sources into the patient's mouth. The lights can be held in close proximity to the composite resin materials residing in the patient's mouth. The exposure times required for curing the composite materials depend on the types of composite resins used. Thus, the lighter the handhelds, the easier it is for the dental professionals who have to hold such devices in place to effect curing.

### SUMMARY OF THE INVENTION

The present invention relates to a portable dental curing light suitable for curing light curable dental composite materials with a high density, uniform and convergent light. The curing light device may include a housing having a handle portion towards its distal end, a front portion towards its proximal end, and a light module inside the front portion. The light module houses one or more light sources, which may include, for example, light emitting diodes (LEDs). The LEDs may emit wavelengths having a single or multiple peaks, one or both is in the range of most photosensitizers used in the curable composites such as dental composites.

The light source may generally be arranged to produce a convergent light beam. The converged light may minimize heat generation more effectively to increase the runtime of the device. Furthermore, if heat may be minimized, the size and costs of elaborate cooling system may be minimized, so as to enable a lighter, more portable handheld curing light device.

One or more optical elements may be disposed in front or about the light sources to aid in directing or focusing the light emitted from the light sources, for example, LEDs, to form a more convergent beam, and at least one heat sink located inside the light module to conduct heat away from the light source. The optical element may be made of any substantially transparent material. Most light sources emit light in a cone and any light outside of any light capturing device is wasted if allowed to escape. The edges of the optical element of the present invention may be reflective so that light hitting the edges may be reflected back into the path of useful light.

The housing of the curing light may be substantially cylindrical, with a slight taper from the distal end to the proximal end, for example. The proximal end of the housing may be straight or may be angled.

The proximal end of the housing may further include a lens cap, to close off the proximal end of the curing light so as the keep the light module from coming into contact with the patient's mouth.

In general, the lens cap is transparent. In one embodiment, the proximal end of the curing light includes a housing formation for inter-engaging a cap formation on the lens cap. In one aspect, the lens cap may only serve as an exit for light from light source or sources, for example, does not substantially transform the intensity or wavelength of the light from the light source, and/or a protective cover to close the light emitting end of the curing light, as noted above. In another aspect, the lens cap may have other optical properties to also serve to focus the light from light source.

In an exemplary embodiment, the lens cap may be substantially circular and includes a top surface, a bottom surface and a cap formation somewhere between the top and bottom surface about its peripheral. The cap formation may include a peripheral groove about midway located between the top surface and the bottom surface and extending about the periphery; and the housing formation may be a protrusion or a receiving edge, extending about its peripheral for matting with the groove in the lens cap, so that the lens cap may be snapped onto the proximal end of the housing by slightly and inwardly pressing these two inter-engaging formations of the housing and lens cap.

The shape and size of the peripheral groove and that of the receiving edge are complementary, such that the peripheral groove can perfectly engage with the receiving edge when the lens cap snaps on the light emitting end, closing off the proximal end of the housing.

In one embodiment, the peripheral groove extends more than half of the periphery of the lens cap to create a longer engagement portion between the lens cap and the proximal end of the housing. This “over-centered” engaging scheme provides a more secured engagement therebetween, and a better protection of the optical instruments therein on the one hand, while the lens cap may be easily detached from the housing. This detachment may be effected by applying a force to the top surface of the lens cap, drawing the lens cap away form the proximal end to disengage the peripheral groove and the receiving edge. For example, the user may apply a force on the top surface of the lens cap in the direction substantially perpendicular to the direction of light coming out of the light source, thus drawing the lens cap away from the proximal end to disengage the “inter-engaging” formation of the housing and the lens cap. Pushing the peripheral of the lens cap against a hard surface may also effect such disengagement. This gives the user the secure feeling that the lens cap is not likely to become lost or become loose in the patient's mouth during a procedure, while allowing easy removal for cleaning.

In another embodiment, the lens cap may include a pair of engaging units adapted to correspondingly engage with a pair of receiving slots located on the proximal end of the housing when the lens cap is snapped thereon to provide more secure attachment.

In other embodiments, internesting pin and pinhole formations, latches and other interconnecting structures are also contemplated.

The lens cap may also be disposed off if it becomes too dirty or has too many scratches on the surface.

The housing of the curing light may have a substantially hollow interior with at least one heat sink located therein, as discussed above. The heat sink may take on various shapes, and/or may include at least one phase change material, some of which may facilitate the arrangement of the light sources for a longer runtime device.

In an exemplary embodiment, the light module may include a light source having at least three LEDs arranged on a substrate, in the shape of a triangle, and may also be an equilateral triangle, for example, with an optical element adapted for directing and focusing the light from one or all of the LEDs for light emitting outside the optical element, as noted above. The substrate may include alignment formations. In one aspect, the optical element includes at least three separate lenses, when three light devices or LEDs are arranged in a triangular arrangement, as noted above. In another aspect, the optical element includes at least three lenses, substantially connected, each adapted for directing and focusing the light from one of the LEDs.

In other exemplary embodiments, any number of LEDs may be present and arranged in a compact manner, such as arrays or groups of LEDs in multiples of three or other numebrs.

In one aspect, the optical element substantially aligns with the LEDs to direct and focus the light emitted therefrom. For example, the LEDs may substantially align with the lens located on the optical element to generate a convergent and uniform light to more effectively cure a composite material. The optical element may include an optical concentrator for concentrating light in a certain direction. In another aspect, the optical element may have reflective edges that may help to redirect light emitting outside the light path useful in the curing light, as noted above. The reflective edges may also be present in the case where substantial alignment is effected.

In one embodiment, a plurality of alignment legs may be present, extending downwardly from the top surface of the optical element. These alignment legs may be used to plug into the alignment holes on the substrate noted above to precisely align the lenses on the optical element with the corresponding LEDs. In one aspect, each alignment leg may include a locking element to engage with the edge of either the mounting surface of the heat sink, or the alignment hole of the substrate.

In one embodiment of the invention, the lens cap, the optical element and the portion of the proximal end of the housing to which the optical element and lens cap are attached may be made of the same material or material having substantially similar coefficient of thermal expansion.

The above has been described in terms of LEDs as light sources. Light sources may include semiconductor light emitting devices, light-emitting chips such as an LED, a solid state LED, an LED array, edge emitting chips, and so on. In another aspect, any number of LEDs may be present and arranged in a compact manner.

In one embodiment, the light sources, e.g. LEDs, may be disposed in the head portion of the curing light in a compact manner. In one aspect, the light source is arranged to generate a high intensity round beam. In another aspect, a high intensity square beam is generated.

The present invention also relates to a dental curing light suitable for curing light curable dental composite materials. The curing light includes a light module housing having a distal end and a proximal end. The light module housing may have a substantially cylindrical shape defining a substantially hollow interior, a handle, a head and neck portion, with at least one elongated heat sink located therein. The head portion may be angled with respect to the rest of the housing. At least one mounting surface is located towards the proximal end of the elongated heat sink.

In one embodiment, located, positioned or mounted on the mounting surface is a light source, for example, three LEDs arranged in a triangular pattern, emitting uniform and convergent light of a wavelength suitable for promoting cure of a composite material.

In other embodiments, more than three LEDs are present and are arranged in other patterns such as in rectangle, in hexagonal, and so on. The number and arrangement of the LEDs depend upon the size of the head portion of the curing light.

A thermistor may also be present to provide temperature control of the light source wherein one end of the thermistor is attached to a control circuit board, and the other end may be inserted into the heat sink. When the temperature of the LEDs is higher than certain set point of the thermistor, a signal to shut off the curing light may be transmitted to the control circuit board to cool down the curing light for a period of time, which may not only prevent the curing light device from overheating, but may also protect patients from discomfort.

In one embodiment, the mounting surface may include an optical element, which may be concave or convex, and for directing and/or focusing light from a light source to a desired location, such as the mouth of the patient. In one aspect, the optical element may include lenses that are individually aligned with the corresponding LEDs to direct and/or focus light therefrom, to generate a high intensity round beam with less heat dissipation.

In another embodiment, the curing light may include an internal connector adapted to connect the handle portion and the heat sink. In one aspect, the internal connector may serve to limit the rotational movement of the heat sink by aligning an opening on the internal connector with a corresponding opening located on an engaging portion of the heat sink. If the two openings are not aligned with each other, the heat sink disengages the internal connector.

In a further embodiment, a guiding slot located at the side wall of the front portion of the heat sink matches with a guiding protrusion inside the front portion. When the heat sink is slid into the hollow interior of the front portion, the guiding slot matches with the guiding protrusion to guide the substrate to an appropriate position.

In one aspect, a heat sink made of a solid block of thermally conductive material, such as metal, may be used to efficiently remove or divert heat from a light source or sources. In another aspect, the heat sink may be configured to have fins, corrugations, or other geometric features adapted to provide a larger surface area for convective cooling of the heat sink. In a further aspect, the heat sink may include a substantially hollow interior which may be partially filled with at least one suitable phase change material including organic materials, inorganic materials and combination thereof, as noted before. These materials can undergo substantially reversible phase changes, and can typically go through a large, if not an infinite number of cycles without losing their effectiveness. A capping device may be used to cap off the heat sink after filling with the phase change material. The capping device may be compression fit. Any fitting may be sufficient to withstand any expansion and/or contraction force during cycling of the phase change material. In still another aspect, the heat sink may be constructed by hollowing out a thermally conductive material, such as metal, and at least partially filling the space with at least one phase change material prior to capping it to secure the phase change material inside, such that at least one phase change material is substantially contained or surrounded by a thermally conductive material such as a metal normally used in the construction if a conventional metal heat sink.

In the above mentioned aspects, the mounting of the light source is away from the capping device.

The present invention further relates to a dental curing light having a light source arrangement and an optical element combined to provide a larger effective beam diameter and surprisingly slower fall off in power as the distance between the emitting end of the curing light and the target for restoration increases. The light module housing includes a distal end and a proximal end, an elongated heat sink and at least one optical element formed on or attached to at least one light source, in any of the embodiments described above. The light module housing may have a substantially cylindrical shape defining a substantially hollow interior, a handle, a head and neck portion, which may be angled with respect to the rest of the housing. At least one mounting surface is located towards the proximal end of the elongated heat sink. The light source may be located at the distal end of the heat sink, and if a phase change heat sink is used, away from the capping device.

The curing light of the present invention is compact, light weight and portable.

In one aspect, the optical element may include a reflector, which may be, for example, of a parabolic shape, capable of directing the light emitted by the light source towards the proximal end or head and neck portion of the light module housing. In another aspect, a mounting platform may be present on the heat sink and the platform may include an optical element. In a further aspect, the optical element may include multiple components adapted for capturing substantially all the light emitted by the light source located or positioned at the distal end of the heat sink by redirecting any light outside of the path.

A larger effective beam diameter has the advantage that fewer exposures may be needed to cover a specified area of the tooth. For example, when the restoration area exceeds a beam diameter, the clinician provides multiple overlapping exposures to the area in order to completely irradiate the restoration. With a larger beam diameter, the clinician need not move the tip to cover the entire area. Also, if insufficient power is generated, the clinician merely needs to stay in place to deliver longer exposure duration.

The integrated power output of the curing light is generally highest at the emitting end or tip of the curing light. The integrated power output constitutes an effective beam diameter. The effective beam diameter generally varies with distance. However, dentistry is rarely performed at a zero-distance from the emitting end or tip of the curing light to the restoration being light-cured. The dispersion of the beam as the exit tip distance to the target is increased is of great clinical importance. Most beam diameters are small to insure that the power fall off with distance is small because the beam divergence of a larger beam diameter is generally greater than that of a smaller effective beam diameter. In other words, and as stated previously, having a larger beam diameter may be a benefit clinically, speaking in terms of efficiency, however, the power in this large beam diameter over the ever increasingly large beam area does not increase, and may lead to a very large fall in power density with increasing emitting end distance. The present invention surprisingly limits the loss in output power to be in the range of a smaller effective beam diameter.

In general, all curing lights may have a non-uniform distribution of power density within a generated beam, large or small. The presence of very intense areas and relatively low intensity locations are generally present. Composite will cure in direct correlation with the local power levels delivered. The conversion process does not spread out laterally to help homogenize the overall curing.

In one exemplary embodiment, the light source may include three light devices, such as LEDs, arranged in a triangular arrangement. In one aspect, the arrangement may be an equilateral triangular arrangement.

In another exemplary embodiment, the light source may include more than, for example, three-LED in an arrangement of other shapes.

In a further exemplary embodiment, the light source may include, for example, multiples of three-LED in an equilateral triangular arrangement.

A triangular shape arrangement shows its greatest power density emission within the center of the beam, and with much lower levels inside and outside of that triangle. However, surprisingly, within most of that beam, the output is of a fairly homogeneous light green color. This homogeneity continues for a distance away from the emitting end, of at least 2 mm away, for example, in one aspect. In another aspect, the uniformity continues at least 4 mm away. In a further aspect, the uniformity continues up to at least 6 mm away.

Any of the curing lights described above may provide light at single or multiple wavelengths commonly used for restorative compounds, as noted above. In one aspect, for multiple wavelengths, the LEDs may emit multiple wavelengths. In another aspect, the LEDs may emit one wavelength and the optical element may include a wavelength transformer having a chemical capable of absorbing the incident light and emitting light having a longer wavelength.

The curing lights of the present invention may include a light transport device at the proximal end of the housing. In one aspect, the light transport device may be a light guide, which may envelope the lens cap disclosed above. In another aspect, the light transport system may be a focusing dome that may be the lens cap described above or in addition to the lens cap described above and may also be capable of varying the beam diameter of the light exiting the curing light device. In a further aspect, the light transport system may be a tacking tip.

The curing light may be un-tethered and powered by, for example, a portable energy source, such as a battery, capacitor and/or combinations thereof. A charger may be provided for charging the portable energy source during off cycle. In one aspect, the charger base may include an electric motor mechanically coupled to a fan or turbine. The fan or turbine may be adapted to draw or urge ambient air across a surface of the heat sink to provide cooling of the heat sink. In one embodiment, this cooling may occur when the curing light is at rest or being recharged. In another embodiment, the cooling means is present inside a charger base or cradle, for recharging the curing light. In other embodiments, the charger base or cradle may not have a fan or cooling means, but instead or additionally, many include a display panel for displaying a condition of the battery.

In an exemplary embodiment, the electrical current provided to the LEDs may be in a pulsed manner to improve heat dissipation. It is surmised that pulsing may increase the “runtime” of the curing light and enhance the performance of the LEDs. In one aspect, a pulse circuit is used to generate a pulsed electrical input to create a pulse LED output. In another aspect, the pulse circuit may include a microprocessor. In a further aspect, the pulse circuit may include an input for receiving signals from the thermistor and an output for controlling the intensity of the LEDs to prevent heat buildup. In still a further aspect, the pulse circuit may monitor the output power level of the LEDs and make corresponding adjustment of the electrical current to prevent heat accumulation, and further improve the runtime of the curing light device.

In one aspect, the pulsing may be effected by varying the input power to the LEDs. In anther aspect, the pulsing may be effected by controlling the output from the LEDs.

The present invention together with the above and other advantages may best be understood from the following detailed description of the embodiments of the invention illustrated in the drawings below.

### BRIEF DESCRIPTION OF THE DRAWINGS

FIG. 1 illustrates a perspective view of a curing light of the present invention.

FIG. 1a illustrates an exploded view of the curing light of the present invention.

FIG. 1b illustrates a sectional view of the curing light of the present invention.

FIGS. 1c and 1c1 illustrate cross-sectional views of a light module of the curing light in the present invention.

FIG. 1c2 shows a schematic view of the optical element portion of the light module of FIGS. 1c and 1c1. FIG. 1c3 shows an exploded view of the optical element portion of the light module of FIG. 1c2.

FIG. 1d illustrates an exploded view of the heat sink and portion of the light module attached to the heat sink.

FIG. 1e illustrates a perspective view of the optic element of the curing light in the present invention.

FIG. 1f illustrates a substrate attaching to the mounting surface of the heat sink.

FIGS. 1f1 and 1f2 illustrate exemplary embodiments of a triangular arrangement.

FIG. 1g illustrates a posterior view of the lens cap.

FIG. 1h illustrates a lateral view of the lens cap.

FIG. 1i illustrates a perspective view of the lens cap engaging with the light emitting end with a pair of engaging units.

FIG. 1j illustrates a lateral view of FIG. 1i.

FIG. 1k illustrates a sectional view of a substrate on which the LEDs are mounted in the present invention.

FIG. 1l illustrates a scheme to remove the lens cap from the proximal end of the front housing.

FIG. 2 illustrates a perspective posterior view of the handle portion of the curing light in the present invention.

FIG. 3 illustrates a perspective anterior view of the handle portion of the curing light in the present invention.

FIGS. 4 and 4a illustrate a charger base of the curing light in the present invention.

FIG. 5 illustrates a perspective view of the curing light with a pulse circuit.

FIG. 5a illustrates the pulse circuit electrically connecting with the thermistor and the LED circuit.

FIG. 6 shows a perspective view of an embodiment of the present invention including a light transport.

FIG. 7 shows a cross-sectional view of an embodiment of the emitting end of the light module housing fitted with a lens cap and a light transport of the present invention.

FIG. 8 shows the change in effective beam diameter with increasing emitting end-to-target distance of representative commercially available curing lights.

FIG. 9 shows the change in power density with increasing emitting end-to-target distance for representative commercially available curing lights.

FIG. 10 shows a Top Hat Factor.

FIG. 11 shows the power density distribution across the emitting chip assembly (no lens present).

FIG. 12 shows the power density distribution across the emitting end of the curing light of the present invention.

FIG. 13 shows Top Hat Factor for an embodiment of the present invention at various distances from the emitting end.

FIG. 14 shows the change in effective beam diameter with increasing emitting end-to-target distance of an embodiment of the present invention.

FIG. 15 shows the change in power density with increasing emitting end-to-target distance for an embodiment of the present invention.

FIG. 16 shows a cross-sectional view of an arrangement of LEDs used in the uniformity measurement.

FIG. 16a shows a cross-sectional view of the enlarged substrate portion of FIG. 16.

FIG. 16b shows an optical element used in the example as an embodiment of the present invention.

FIGS. 17a, 17a1, 17b, 17b1, 17c, 17c1, 17d and 17d1 show various substrate designs for heat sink attachment.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplary methods, devices and materials are now described.

All publications mentioned herein are incorporated herein by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

A curing light device useful for curing or activating light-activated materials is disclosed. The present invention has applications in a variety of fields, including but not limited to medicine and dentistry, where light-activated materials including a photoinitiator or photoinitiators are used. As an example, a photoinitiator absorbs light of a particular wavelength and initiates the polymerization of monomers into polymers.

In an exemplary embodiment, light-activated materials including a single photoinitiator or multiple photoinitiators may be applied to a surface, such as a tooth surface, and later cured by light of a wavelength or wavelengths that activates or activate the photoinitiator or photoinitiators. The light used is not only of a wavelength to which the photoinitiator is sensitive, but also of a power level adapted to cause curing over certain durations of time. Although the light used to activate the photoinitiator is of a wavelength to which a photoinitiator is sensitive, the light may come from a variety of sources, for example, a lamp, an arc lamp such as a halogen light source, semiconductor light emitting devices, light-emitting chips such as an LED, a solid state LED, an LED array, a fluorescent bulb, and so on. Further for example, the present invention comprises light sources including semiconductor chips, LED dies, solid state LEDs, LED arrays, edgec emitting chips, or combinations thereof. The light source may include an emitting surface or at least one emitting edge as in the case of an edge emitting chip noted above, for a compact curing light device.

The typical sensitizers used in composite curing include Camphorquinone (CQ), which absorbs at about 465 nm and phenyl-propanedione (PPD), which absorbs at about 390 nm. Dental curing lights having multiple wavelengths suitable for curing curable composites usually comprise output wavelengths encompassing both of the absorbing wavelengths of these two typically used photo-initiators. The output wavelengths generally include a composite spectrum generated by LEDs or LED arrays emitting different wavelengths. The present invention comprises a curing light capable of curing all typical dental composites using, for example, light sources mentioned above, including semiconductor chips, LED dies, solid state LEDs, LED arrays, edge emitting chips, or combinations thereof, mounted on mounting platforms configured on at least one heat sink.

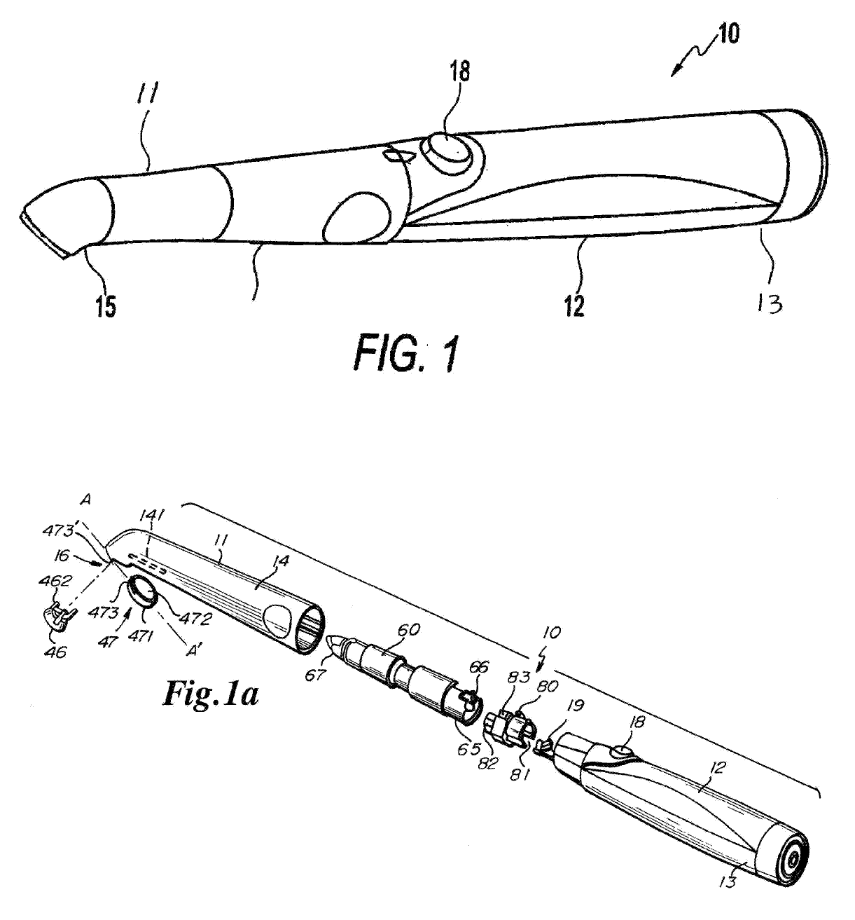

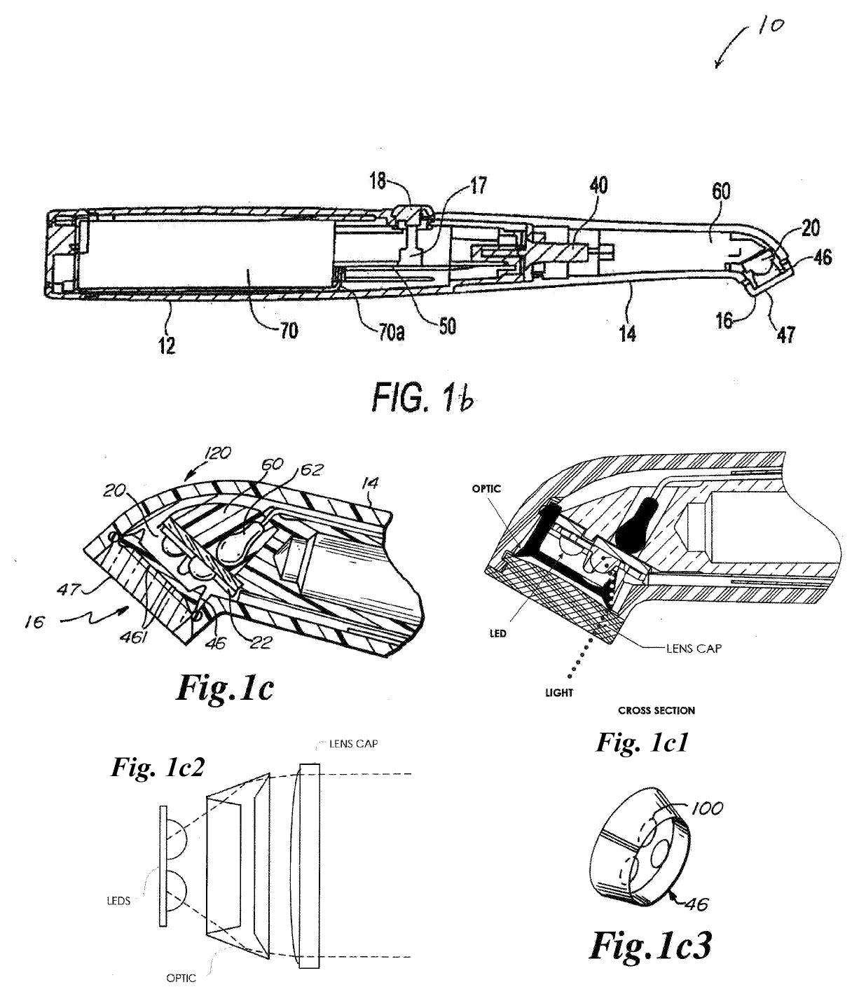

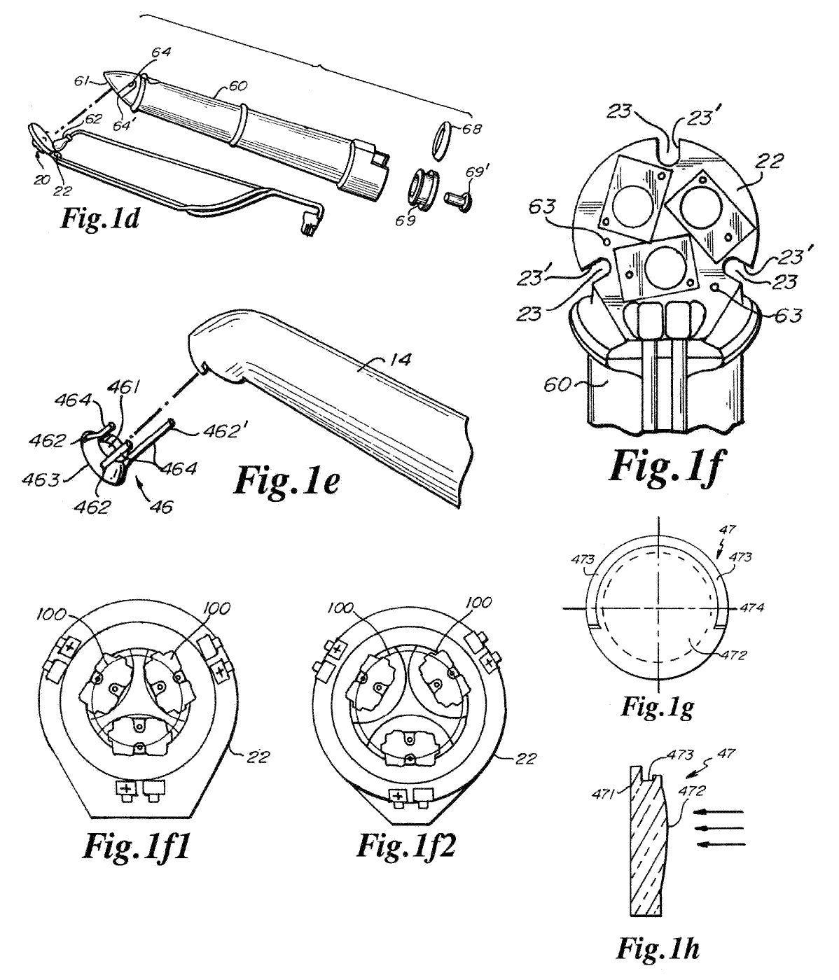

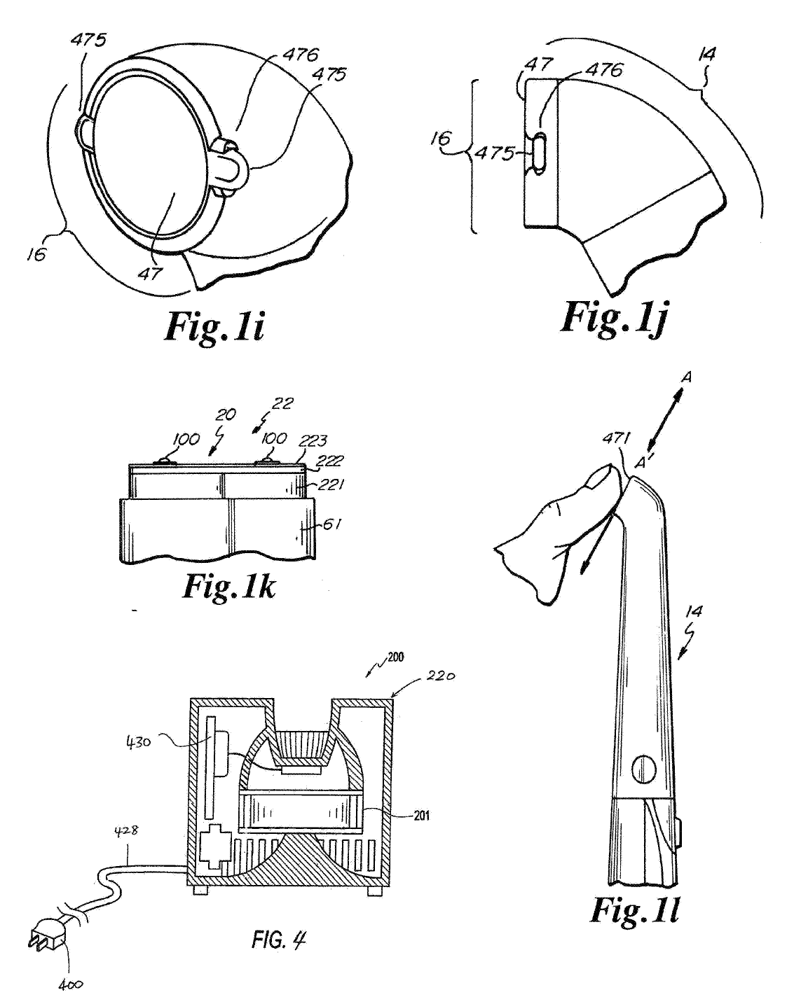

According to one embodiment, as illustrated in FIG. 1, 1a or 1b, a handheld curing light 10 of the present invention includes a longitudinal housing having a distal end 11 and a proximal end 13 with a substantially hollow interior. In the present embodiment, the housing may include two portions, as depicted in the figures, a handle portion 12 and a front portion 14. It is noted, however, that a one-part housing may also be contemplated as part of the present invention. The front portion 14 may also be an extension of the housing, especially if an integral housing is present. A light transport portion or other extensions may also be included as part of the present invention, as shown in FIG. 6.

The portions 12 and 14 may be joined together by any attachment means, with the proximal end of handle portion 12 abutting the distal end of the front portion 14. Suitable attachment modes include, but are not limited to, friction fit, mating bayonet formations, tongue and groove type formations, internesting pin and pinhole formations, latches and other interconnecting structures.

As shown in FIG. 1, front portion of housing 14 of the curing light of the present invention also has a neck section 15, and this neck portion may be configured such that an emitting end 16 substantially coincides with the terminal end of the mounting deck, surface, platform or member of the light source 20, as shown in FIG. 1b. The neck portion may also be angled with respect to the longitudinal portion of the housing.

In one embodiment, as shown, for example, in FIG. 1c, the front portion of the housing 14 may include a light module 120 in a desirable position in the interior of the front housing portion 14. The light module may include at least one light source 20, which, for example, may include three light sources or three LEDs 100 or three dies 100 arranged on a substrate 22, as shown in FIG. 1k. The arrangement may be in the shape of a triangle or an equilateral triangle. An optical element 46 having at least three separate lens 461, as shown, or 100a, as shown in FIG. 1c3, may be adapted for directing and focusing the light from the LEDs. In another embodiment, the lens 461 may be inter-connected to form a continuous structure. At least one heat sink 60 may be located inside the light module 120 to conduct heat away from the light source 20. The proximal end 14 of the housing may further include a lens cap 47, which may be transparent, as exemplified, to provide an exit aperture for light from light source 20 and to serve as a protective cover to close the light emitting end 16 of the curing light. The lens cap 47 may also include other optical properties for focusing the light, etc., as discussed above, and in more detail below.

The optical element 46 may be made of any substantially transparent material, including materials such as polycarbonate (Lexan®), polyacrylics, or any of the materials mentioned below that is substantially transparent.

As mentioned above, most light sources emit light in a cone and any light emitting outside of any light capturing device, such as the optical element 46, is wasted if allowed to escape. The optical element 46 may include reflective edges which is capable of redirecting light. This is shown in cross-sectional view in FIG. 1c1 where the edge reflection and redirecting is shown by the dots. This is more clearly shown in the cross-sectional view in FIG. 1c2, where the dotted lines show how the light hitting the edges of the optical element 46 is redirected.

FIG. 1c3 shows a perspective view of the optical element 46 portion of the cross sectional view of FIG. 1c2. The lens or lenslets 100a, or 461, as shown in FIG. 1c, may be fitted over the LEDs in a three LED arrangement of an exemplary embodiment. The lens 100a may be separate, as shown here, or they may be connected, as discussed further below.

In an exemplary embodiment, the lens cap 47 may be substantially circular and includes a top surface 471, a bottom surface 472 and a cap formation 470 somewhere between the top and bottom surface about its peripheral. The cap formation 470 may include a peripheral groove 473 about midway located between the top surface 471 and the bottom surface 472 and extending substantially about half of the periphery along the peripheral; and the housing formation may be a protrusion or a receiving edge 473′, extending also about half of its peripheral along the peripheral, for matting with the periphery groove 473 in the lens cap 47, so that the lens cap 47 may be snapped onto the proximal end of the front housing 14 by slightly and inwardly pressing these two inter-engaging formations of the housing and lens cap 47 together.

In FIG. 1a, a receiving edge 473′ is located at the light emitting end 16 of the proximal end 14 of the housing. The shape and size of the peripheral groove 473 of the lens cap 47 and that of a receiving edge 473′ located at the light emitting end 16 are complementary, such that the peripheral groove 473 may perfectly fit with the receiving edge 473′ when engaged with each other, as can be seen in FIG. 1a, to form a secure attachment. In one embodiment, the peripheral groove 473 extends more than half of the periphery, creating an “over-centered” engaging scheme that provides a longer engagement portion between the lens cap 47 and the light emitting end 16 to create a more secured engagement therebetween, and may further provide a better protection of the optical instruments therein even if the fit between the groove 473 and receiving edge 473′ is not perfect.

The advantage of this attachment is also to provide an easy detachment scheme when the lens cap 47 needs to be replaced. The lens cap 47 may be easily detached from the light emitting end 16 of the curing light 10 when force is applied on the top surface 471 at certain directions to draw the lens cap 47 to disengage the peripheral groove 473 and the receiving edge 473′. For example, the user may apply force on the top surface 471 of the lens cap 47 at a direction A-A′ substantially perpendicular to the light emitting from the light source 20 to draw the lens cap 47 down to disengage the “inter-engaging” formation of the front housing portion 14 and the lens cap 47, as illustrated in FIGS. 1a and 1c. This gives the user the secure feeling that the lens cap is not likely to become lost or become loose in the patient's mouth during a procedure, while allowing easy removal for the user to clean the lens cap 47 and the optical element 46.

In another embodiment, as exemplified in FIGS. 1i and 1j, the lens cap 47 may include a pair of engaging units 475 symmetrically located at the periphery of the lens cap 47, to be connected to a resilient material, so that the user may slightly press the engaging units 475 inwardly to engage the receiving slot 476 located at the light emitting end 16.

In other embodiments, internesting pin and pinhole formations, latches and other interconnecting structures are also contemplated for interconnecting the lens cap and the housing at end 16.

In one aspect, the lens cap 47 may have no optical properties and is simply a protective cover, as noted above, and the light existing from the housing end or emitting end 16 may pass through the lens cap 47 without undergoing any optical changes. In another aspect, the lens cap 47 may be a focusing lens, a dome or similar, adapted to focus the light from the LEDs. As exemplified in FIG. 1h, the bottom surface 472 of the lens cap 47 receives the light coming from the LEDs. The bottom surface 472 may be slightly convex so that the light may be focused to a certain extent by the convex bottom surface 472 before reaching the top surface 471. In other aspects, the top surface 471 of the lens cap 47 may have optical properties or both surfaces may have optical properties.

In one aspect, the lens cap 47 and the portion of the emitting end 16 that mates with the lens cap 47 may be made of the same material. In another aspect, the lens cap 47 and the portion of the emitting end 16 that mates with the lens cap 47 may be made of different materials having similar coefficients of thermal expansion.

In an exemplary embodiment, as shown in FIG. 6, the lens cap 47 may be covered by a light transport device 175, such as a light guide, a focusing lens, a tacking tip, or similar structure for molding, shaping or compacting the curable composite or for focusing the light source further or combinations thereof, if desired. The device 175 may be a discrete attachment adapted to be coupled to the lens cap 47.

In one embodiment, for example, the device 175 may be adapted to be securely fitted over the end 16 of a curing light device until actively removed, without the need to remove the lens cap 47 of the present invention that may be mislaid or lost. In one aspect, the light transport device 175, such as a tacking tip, may attach itself to the lens cap 47 by surrounding it and covering by snapping them together. They may be held together by frictional force. The transport device may also have an enlarged outer diameter portion 160b, if desired, to facilitate gripping or positioning of the tip 175, as shown in FIG. 6, to be discussed more below.

In another aspect, the attachment between the lens cap 47 and the transport 175 may be aided by a bump, a ridge, a rim, a protrusion or similar raised structure 220 included on the outside of the lens cap 47 with a corresponding groove, channel, depression or enlarged inner diameter portion 130 of tip 175 on the inside of the tip 175 such that the locking interaction between the ridge or rim 220 on the lens cap 47 and the corresponding groove, channel, depression or enlarged inner diameter portion 130 of tip 175, as shown in FIG. 7. The mating of ridge or rim 220 of the lens cap 47 with the enlarged inner diameter portion, depression, channel or groove 130 of the enlarged portion 160b of the tip 175 may also serve to position the tip 175 onto the lens cap 47 in a repeatable fashion. Similarly, the position of the channel, depression or enlarged inner diameter portion 130 may also serve to vary the pre-determined position of the tip 175.

In a further aspect, not shown, the bump, ridge, rim, protrusion or similar raised structure may be included on the tip 175 with a corresponding groove, channel, depression or similar feature included on the lens cap 47 so as to provide a substantially similar positioning feature as discussed above.

In yet a further aspect, a threaded attachment may be used.

In another embodiment, the device 175 may have a formation that may mate with the emitting end 16 of the curing light in place of the lens cap 47. This attachment may be also effected by frictional force, the mating mechanism, or a threaded attachment, discussed above.

Referring to FIG. 7, an aperture 120 is disposed towards the distal end 140, near the apex of the device or tip 175. In one embodiment, apart from the aperture 120, the tip 175 is substantially opaque to light emitted by the curing light 10. Thus, the diameter or footprint of the beam exiting the curing light device corresponds to the diameter or size of the aperture 120 of the tip 175. In another embodiment, a series of tips 175 with varying aperture size is envisioned for use depending on the size of spot curing desired to add versatility to the system. All the tips 175 in a series may be sized to fit securely over a lens cap 47 and for the same curing light, only the size of the aperture 120 is varied.

In one embodiment, at least portions of this interior of the tip may be reflective, for reflecting, directing or focusing light entering the tip 175 from the curing light towards the aperture 120. In another embodiment, the interior may be opaque, adapted to absorb light except that passing through the aperture 120.

The aperture 12 may be substantially smaller than the diameter of the interior of the rest of the tip 175, serving to reduce the footprint of the light exiting the tip 175 in relation to the light entering it, effecting spot curing even if the diameter of light exiting the lens cap 47 is relatively large.

The tip 175 also may have an elongated portion towards the distal end 140 and aperture 120 that may also allow for spot manipulation of the curing compound, if desired.

In one aspect, the portion of the device 175 that envelopes the portion of the lens cap 47 may be made of the same material. In another aspect, the portion of the device 175 that envelopes the portion of the lens cap 47 may be made of different materials having similar coefficients of thermal expansion.

Generally, a larger effective beam diameter i.e., the diameter of the beam exiting the emitting end 16, has the advantage that fewer exposures may be needed to cover a specified area of the tooth, as noted above. For example, when the restoration area exceeds a beam diameter, the clinician provides multiple overlapping exposures to the area in order to completely irradiate the restoration by aiming the curing light at various locations within the area. This can be time consuming and may also lead to both over curing and under curing. With a larger beam diameter, the clinician need not move the tip, which may be the emitting end 16 or the tip 175, to cover the entire area. Also, if insufficient power is generated, the clinician merely needs to stay in place to deliver longer exposure duration without having to aim the beam at various locations.

Most commercially available curing lights also have small effective beam diameters to insure that the power fall off with distance is small because the beam divergence of a larger beam diameter is generally greater than that of a smaller effective beam diameter. In other words, and as stated previously, having a larger beam diameter may be a benefit clinically, speaking in terms of efficiency, however, the power in this large beam diameter over the ever increasingly large beam area does not increase, and thus may lead to a very large fall in power density with increasing emitting end distance from the target. Hence, over producing power output and small effective beam diameter are the norm.

Of course, the clinician with a curing light having a narrow beam may also hold the emitting end 16 at a larger distance away from the restoration area. FIG. 8 shows an exemplary graph of how the beam size increases with distance away from the emitting end 16 for representative commercially available curing lights. However, the power output of most curing lights decreases quite drastically with distance, even with small beam diameters. FIG. 9 shows the change in power density with increasing emitting end 16 to target distance, again for the same representative commercially available curing lights used in FIG. 8. Thus, most commercially available curing lights having a narrow beam tend to over produce power output to compensate for this decrease so that the clinician has the option to hold the curing light farther way from the target to cover a larger area without overlapping illumination. This is not energy efficient. In addition, the heat generated by excessive power output also may cause discomfort to the patient and clinician, and a larger heat sink is usually needed to manage heat production, resulting in a larger and heavier curing light than necessary.

In addition, most curing lights have a non-uniform distribution of power density within a generated beam, large or small, with very intense areas and relatively low intensity locations. Composites will cure in direct correlation with the local power levels delivered. The conversion process does not spread out laterally to help homogenize the overall curing. Thus, uniformity or semblance of uniformity is important, both at short and longer distances from the emitting end.

In one exemplary embodiment of the invention, the effective beam diameter is for example, at least about 9 mm. In another exemplary embodiment, the effective beam diameter is at least about 10 mm. In a further exemplary embodiment, the effective beam diameter is at least about 11 mm.

The light source arrangements may take on any geometry. In one exemplary embodiment, the curing light may include a three-light devices light source, such as LEDs, arranged in a triangular arrangement. In one aspect, the arrangement may be an equilateral triangular arrangement.

In another exemplary embodiment of the invention, the light source may include more than, for example, three-LED in an arrangement of other shapes.

In a further exemplary embodiment of the invention, the light source may include, for example, multiples of three-LED in an equilateral triangular arrangement. In this embodiment, an array or group of LEDS may be used in place of one LED.

In one embodiment of a triangular arrangement, the light sources 100 may be packed in various exemplary embodiments. In one exemplary embodiment, the light sources 100 are close together, as shown in FIG. 1f1, in a tight radial configuration on the substrate 22. In another exemplary embodiment of a triangular arrangement, the light sources 100 may be spaced apart, as shown in FIG. 1f2, in a radial configuration.

As noted before, a triangular shape arrangement, as shown in FIG. 1f, shows its greatest power density emission within the center of the beam, and with much lower levels inside and outside of that triangle. However, surprisingly, for the curing lights 10 of the present invention, within most of that beam, the output is of a fairly homogeneous light green color.

Light uniformity or homogeneity with distance is important so that, when holding a tip at a larger distance from the restoration, the clinician can be assured that all areas of the restoration are being substantially equally irradiated. To measure uniformity, i.e., dispersion versus distance, the Top Hat Factor, as exemplified in FIG. 10, is a method that may be used to mathematically evaluate beam homogeneity—if the beam is totally homogeneous, the THF is unity. As values decrease from one, the discrepancy in power distribution increases. Theoretically, a light may have a totally homogeneous beam, no matter the emitting end distance from the target is. However, in practice, no light has a totally homogeneous beam no matter the emitting end distance from the target is.

Surprisingly, the uniformity or homogeneity with distance of the larger effective beam size of the present invention is maintained over a substantial distance from the emitting end 16, for example, up to about 2 mm away from the emitting end 16, more for example, up to about 4 mm from the emitting end 16, and even more for example, up to about 6 mm away from the emitting end 16. Thus, the curing light of the present invention not only offers a larger effective beam diameter, but also flexibility in distance away from the emitting end.

The light source may include semiconductor light emitting devices, light-emitting chips such as an LED, a solid state LED, an LED array, edge emitting chips, and so on. For an LED, for example, it may include a substrate 22 which may have a plurality of layers, for example, including a copper layer 221 at the bottom of the substrate 22 to directly contact a mounting surface 61 of the heat sink 60, a fiber glass layer 222 in the middle and a printed circuit layer 223 on the surface on which the LEDs are directly mounted. In one aspect, the copper layer 221 is substantially thicker than the fiber glass layer 222 and the printed circuit layer 223, to more effectively conduct heat generated by the LEDs to the mounting surface 61 of the heat sink 60, as shown in FIG. 1k. In another aspect, a more efficient heat sink, such as one partially filled with a phase change material is used.

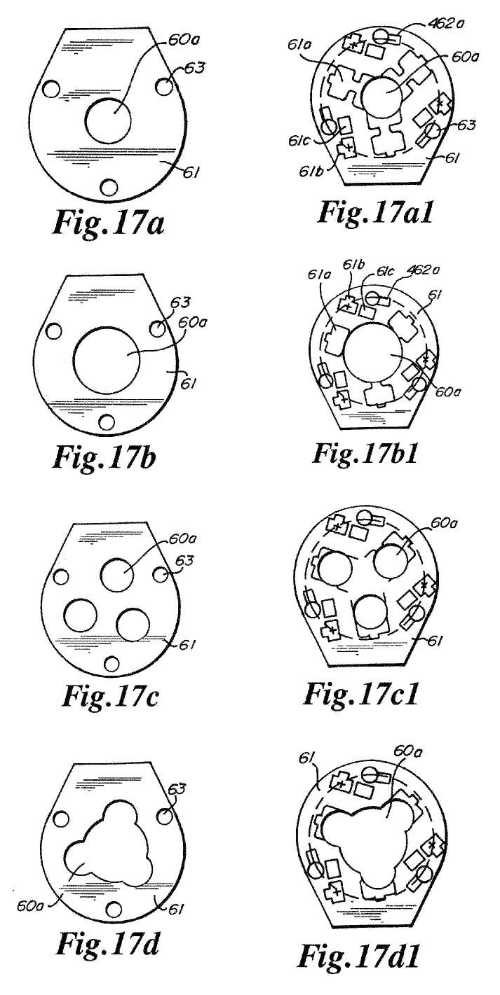

In FIGS. 17a-d, various versions of mounting surface 61 of the substrate 22 with formations for attaching the heat sink 60 are shown. A heat sink 60 may have small bumps or legs to attach to or poke through the substrate. In FIGS. 17a and 17b, a small donut hole 60a or a large donut hole 60a in the middle of the substrate allows a small bump or large bump on the outside of the heat sink 60 to align with the substrate during attachment. In this configuration, solder masks for the thermal pad 61a, anode 61b, and cathode 61c, and an allowance for the optical element 462a are shown in corresponding FIGS. 17a1 and 17b1. The substrate 22 may include various layers as described above. FIGS. 17c and c1 show a design where multiple formations or bumps may be used. In addition, a clover leaf or tri-lobe design may be used, as shown in FIGS. 17d and d1.

In one embodiment, the heat sink 60 may be elongated and positioned inside the front portion 14, in close-proximity to the light source 20, to conduct, or dissipate heat there from. In one aspect, as illustrated in FIG. 1c or 1d, the light source 20 (on the substrate 22) is attached or glued, with thermal adhesive including thermosetting or structural adhesives, to the mounting surface 61 of the heat sink 60. A thermistor 62 may also be attached or glued to the outer surface of the heat sink 60 with the same material noted above, wherein one end of the thermistor 62 may be attached to the control circuit board 50 to control on/off of the curing light 10, and the other end may be inserted into the heat sink 60. When the temperature of the LEDs is higher than certain set points of the thermistor, a feedback signal may be generated and transmitted to the control circuit board 50 to shut off the curing light device 10 for a period of time to bring the temperature down to the set point. This feedback control scheme not only prevents the curing light device 10 from overheat, but also protects patients from discomfort.

To produce an efficient curing light with a compact LED arrangement, in an exemplary embodiment, the LEDs may be substantially precisely align with corresponding lens 461 on the optical element 46, as shown in FIG. 1c. The optical element 46 may further include a plurality of alignment legs 462, extending from the top surface 463 of the optical element 46, as shown in FIGS. 1a and 1e. Each alignment leg 462 may include a locking element 464 adapted for locking the optical element 46 to either the substrate 22 or the heat sink 60. In one embodiment, the heat sink 60 may include at least one alignment pin 63 on the mounting surface 61, and an alignment slot 64 on the outer surface. As shown in FIG. 1f, the substrate 22 may include a plurality of alignment holes 23 on the edge, corresponding to the number and location of alignment legs 462 on the optical element 46. Referring to FIGS. 1d-1f, a two-step alignment scheme is illustrated. First, when the substrate 22 engages with the alignment pin(s) 63 on the mounting surface 61, one of the alignment holes 23 is designed to precisely and automatically align with the alignment slot 64. Second, when the alignment legs 462 plug into the alignment holes 23, the optical element 46 may substantially align with the substrate 22. More specifically, each lens on the optical element 46 may substantially align with the corresponding LED to produce a convergent and uniform round or square beam.

As mentioned before, the lens 100a may be separate, as shown in FIG. 1c3, or they may be interconnected where the lens 100a may be bumps on a continuous surface. For example, the lens may also be formed on a film using micro-replication.

Still referring to FIGS. 1d-1f, one aspect of the invention is illustrated, wherein the optical element 46 may include three alignment legs 462, one of which is longer than the other two. The locking element 464 of the long alignment leg 462′ is adapted to engage an edge 64′ of the alignment slot 64 of the heat sink 60, while the locking elements of the other two short legs 462″ are adapted to engage edges 23′ of two alignment holes 23 on the substrate 22. The length of the alignment slot 64 may be substantially identical to the length of the long alignment leg 462′, and this alignment scheme may secure the optical element 46 more firmly on the substrate 22 to achieve better alignment and further enable the LEDs emitting from the light source 20 to be effectively directed to the desired portion of the patient. In another aspect, the optical element 46 may be an optical concentrator.

The direction of light depends on the shape or curvature of the lenses 461. For example, a concave surface may be used, or a certain degree of curvature of the surface may be designed to influence the direction of the emitting light, individually or collectively. Thus, the shape and the curvature of the lenses 461 will help to shape and direct the light to any desired position.

In one embodiment, the lenses 461 may direct and focus the LEDs to produce a convergent and uniform round or square beam to minimize heat generation to increase the runtime and intensity of the convergent beam of the curing device 10. In another embodiment, after directed and focused by the lenses 461, the beam may be further focused by the lens cap 47 to produce a more convergent and uniform beam.

In other embodiments, more than three LEDs are present and are arranged in other patterns such as in rectangle, in hexagonal, and so on. The number and arrangement of the LEDs depend upon the size of the head portion of the curing light, and the number and pattern of lenses 461 may be arranged to align the corresponding LEDs. In the present invention, a small, compact and light weight curing light 100 is desired, with efficient power usage and good heat management.

It is worth mentioning that a convergent and uniform beam can be effectively generated by the combination of the optical element 46 and the lens cap 47 in the present invention. In other words, by adapting the combination of the optical element 46 and the lens cap 47, a tip 175 externally attached to the light emitting end 16 may not be necessary. Similarly, a reflector may not be necessary so as to reduce the risk of material failure due to differences in the coefficients of thermal expansion between the reflector and the housing 14.

In one embodiment, the optical element 46 and the front portion 14, or at least portions of the front portion 14 may be, for example, made out of the same material, similar material, or different material having little or no difference in the coefficients of thermal expansion. With the presence of different coefficients of thermal expansion, hoop stress may result, which may lead to premature failure of the unit. Such failure is minimized or eliminated by the present embodiment of the invention.

For example, a polymer useful in the present invention may be a polymer that may be molded or cast. Suitable polymers include polyethylene, polypropylene, polybutylene, polystyrene, polyester, acrylic polymers, polyvinylchloride, polyamide, or polyetherimide like ULTEM®; a polymeric alloy such as Xenoy®. resin, which is a composite of polycarbonate and polybutyleneterephthalate or Lexan®. plastic, which is a copolymer of polycarbonate and isophthalate terephthalate resorcinol resin (all available from GE Plastics), liquid crystal polymers, such as an aromatic polyester or an aromatic polyester amide containing, as a constituent, at least one compound selected from the group consisting of an aromatic hydroxycarboxylic acid (such as hydroxybenzoate (rigid monomer), hydroxynaphthoate (flexible monomer), an aromatic hydroxyamine and an aromatic diamine, (exemplified in U.S. Pat. Nos. 6,242,063, 6,274,242, 6,643,552 and 6,797,198, the contents of which are incorporated herein by reference), polyesterimide anhydrides with terminal anhydride group or lateral anhydrides (exemplified in U.S. Pat. No. 6,730,377, the content of which is incorporated herein by reference) or combinations thereof.

In addition, any polymeric composite such as engineering prepregs or composites, may also be used. For example, a blend of polycarbonate and ABS (Acrylonitrile Butadiene Styrene) may be used for the housing. Generally, materials usable in housing include, for example, polymeric materials or composites having high temperature resistance.

A liquid crystal polymer or a cholesteric liquid crystal polymer, such as one that can reflect rather than transmit light energy, may be used in various embodiments of the invention. (For example, in U.S. Pat. Nos. 4,293,435, 5,332,522, 6,043,861, 6,046,791, 6,573,963, and 6,836,314, the contents of which are incorporated herein by reference).

The lens cap 47 is generally transparent, as noted above. Thus, any material that can produce a transparent lens cap may be used.

The lens cap 47 may in general be disposable, so that it may be replaced when it becomes too dirty or has too many scratches on the surface to assure the quality and intensity of the light exiting from the lens cap 47.

Referring to FIG. 1a, the curing light 10 may further include an internal connector 80 which is adapted to connect the handle portion 12 and the heat sink 60. In one embodiment, the handle portion 12 engages with the distal end 81 of the connector 80, while the heat sink 60 engages with the proximal end 82. Furthermore, a control circuit board 50 adapted to control the substrate 22 is received in the hollow interior 81 of the internal connector 80.

It is worth mentioning that the connector 80 may also assist the alignment between the substrate 22 and the optical element 46 by limiting the movement of the heat sink 60. For example, the proximal end 82 of the connector 80 may include an opening 83, which are received in an engaging portion 65 of the heat sink 60. The engaging portion 65 may also include an opening 66 and when the connector 80 engages with the heat sink 60, the openings 66 and 83 are aligned with each other, as shown in FIG. 1a. When the heat sink 60 slightly rotates to misalign the openings, the heat sink 60 disengages the connector 80. In other words, the rotational movement of the heat sink 60 is so limited when engaging the connector 80, such that the precision of the alignment of between the substrate 22 (located at the mounting surface 61 of the heat sink 60) and the optical element 46 is achieved.

In one aspect, a guiding slot 67 is located at the side wall of the front portion of the heat sink 60. The guiding slot 67 matches with a guiding protrusion 141 inside the front portion 14, such that the substrate 22 at the heat sink 60 can be located at the appropriate position when slid onto the guiding protrusion 141, and thus precisely align with the optic element 46.

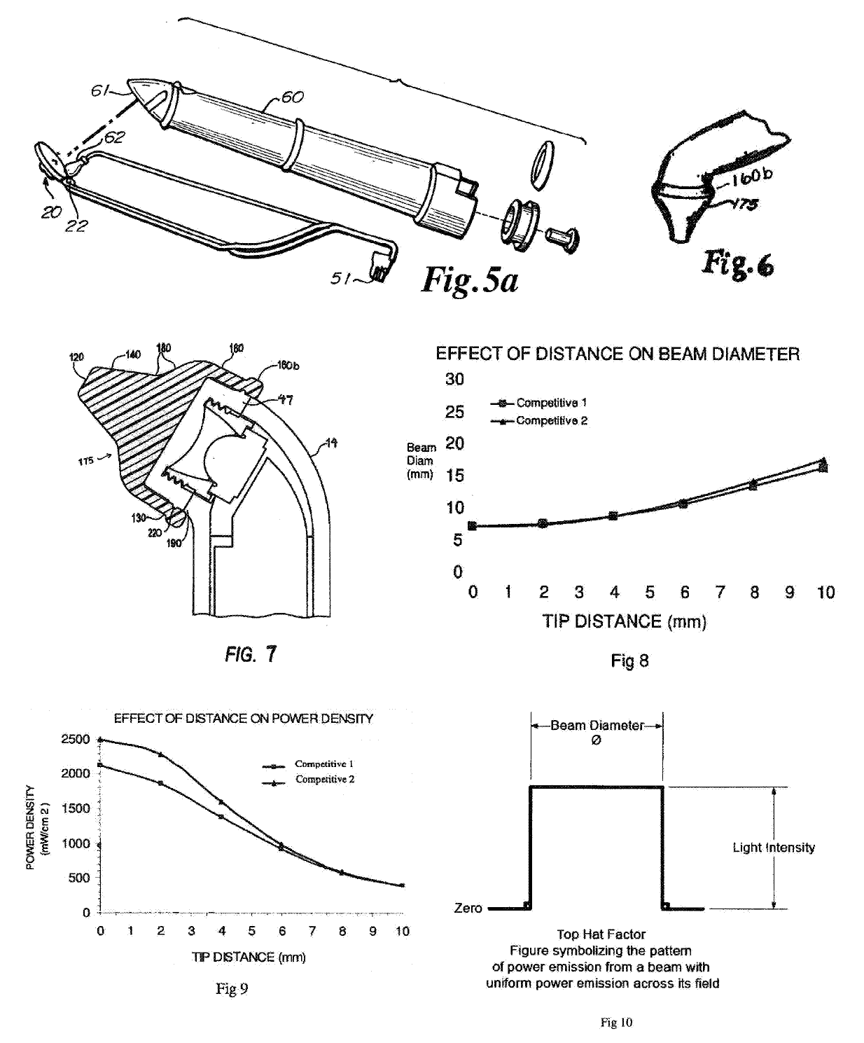

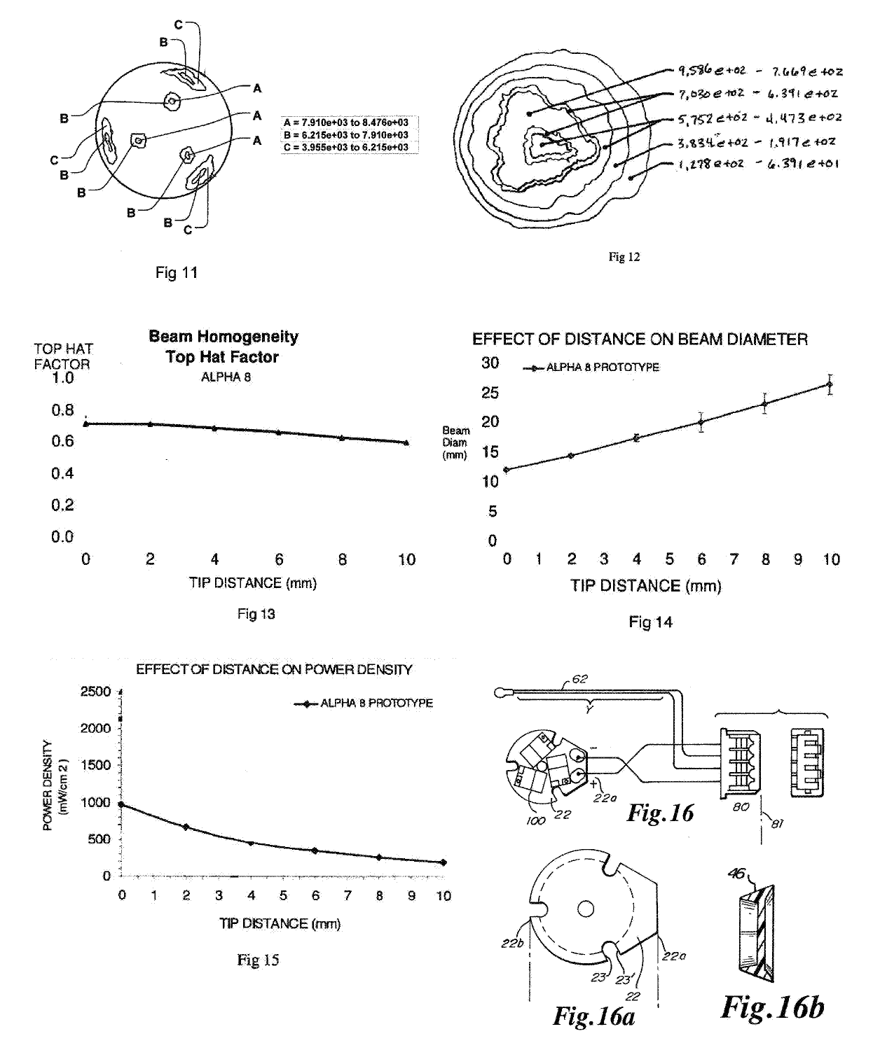

In one aspect, a pulse circuit 51 may provide electrical current to the LEDs in a pulsed manner, meaning that the current is steadily provided for a period of time, and then rest for a period of time, etc, as shown in FIG. 5. When the current is consistently provided to the LEDs, the heat is likely to buildup to decrease the intensity of the lights. If the electrical current is input into the LEDs in a pulsed manner, the heat can be dissipated or released at the time without electrical current. This pulsed current input scheme is surmised to improve the efficiency of heat dissipation, and thus increase the runtime and intensity of the curing light device 10.

In another aspect, the pulse circuit 51 may include a microprocessor, for controlling the on/off cycle, or the input or output power level.

In still another aspect, the pulse circuit 51 may include an input for receiving signals from the thermistor 62 and an output for controlling the intensity of the LEDs to minimize heat generation, as shown in FIG. 5a. More specifically, when the temperature of the LEDs is higher than certain set point of the thermistor 62, a feedback signal may be generated and transmitted to the pulse circuit board 51 to provide a pulse current to the LEDs to minimize the heat generation, as discussed above, and to further increase the runtime of the curing light device 10. It is surmised that the intensity of the curing light device 10 may be increased as well with pulsing.

In a further aspect, the pulse circuit 51 may monitor the output power level of the LEDs in a similar manner as illustrated above, and make corresponding adjustment of the electrical current to minimize heat generation, and further improve the runtime of the curing light device 10.

The pulsing is not limited to on and off, but to different levels of input power or output power. For example, a rest cycle may give off one half of the power of the on cycle, etc. In another example, the pulsing may be a half sinusoidal signal, like a bell-shape curve, going from zero to a maximum and down to zero again, etc.

Generally speaking, the heat sink 60 may be constructed by hollowing out a thermally conductive material, such as metal, and at least partially filling the void with at least one phase change material prior to capping it to secure the phase change material inside, such that the at least one phase change material is substantially contained or surrounded by a thermally conductive material such as metal normally used in the construction of a conventional heat sink.

In another embodiment, the heat sink may include a block of thermally conductive material such as metal having a bore or void space which is at least partially filled with a phase change material.

In a further embodiment, the heat sink 60 may be configured to have fins, corrugations, or other geometric features adapted to provide a larger surface area for convective cooling of the heat sink, whether the heat sink is a solid metallic block, partially filled with a phase change material, so on.

In still another embodiment, the curing light device 10 may include an electric motor mechanically coupled to a fan or turbine (as shown in FIGS. 4 and 4a). The fan or turbine may be adapted to draw or urge ambient air across a surface of the heat sink 60 to provide cooling effect thereof.

The heat sink 60 may be made of any material that has good thermal conductivity, and/or dissipation properties, such as a metal or non-metal, for example, copper, aluminum, silver, magnesium, steel, silicon carbide, boron nitride, tungsten, molybdenum, cobalt, chrome, Si, SiO₂, SiC, AlSi, AlSiC, natural diamond, monocrystalline diamond, polycrystalline diamond, polycrystalline diamond compacts, diamond deposited through chemical vapor deposition and diamond deposited through physical vapor deposition, and composite materials or compounds.

As can be seen in FIG. 1d, the heat sink 60 may further include an o-ring 68, a capping device having a self-sealing screw 69 and a seal cap 69′, wherein the seal cap 69′ is pressed in with the o-ring 68, and the self-sealing screw 69 seals the chamber once the chamber is filled partially with at least one phase change material. The capping device may be compression sealed into the open end, if desired.

Exemplary phase change materials are generally solid at ambient temperature, having melting points between about 30° C. and about 50° C., or between about 35° C. and about 45° C. Also, the exemplary materials may have a high specific heat, for example, at least about 1.7, more for example, at least about 1.9, when they are in the state at ambient temperature. In addition, the phase change materials may, for example, have a specific heat of at least about 1.5, more for example, at least about 1.6, when they are in the state at the elevated temperatures.

Some of the phase change materials mentioned above may be recyclable in that they may undergo phase changes for an almost infinite number of times. Others may be more endothermic agents and thus may have a limited life cycle unless handled under a controlled environment. These endothermic agents may lose their effectiveness as a phase change material even when handled under a controlled environment.

Thermal conductivity of the materials is a factor in determining the rate of heat transfer from the thermally conductive casing to the phase change material and vice versa. The thermal conductivity of the phase change material may be, for example, at least about 0.5 W/m° C. in the state at ambient temperature and at least about 0.45 W/m° C. in the state at elevated temperature.

Heat sinks having a phase change material may more efficiently remove or divert heat from a light source or sources with a given weight of heat sink material when compared to a heat sink made of a solid block of thermally conductive material such as metal. Such a heat sink may even efficiently remove or divert heat from a curing light device when a reduced weight of the material is used. Using a phase change material enclosed inside a hollow thermally conductive material such as a metal heat sink instead of a conventional solid metal heat sink can decrease the weight of the curing light and increase the time the heat sink takes to reach the “shut off” temperature, as it is called in the dental curing light industry. The period prior to reaching the shut off temperature is called the “run time”. Increasing the “runtime”, i.e., the time that the light can remain on, increases the time when a dentist can perform the curing or whitening procedure.

Suitable phase change material may include organic materials, inorganic materials and combinations thereof. These materials can undergo substantially reversible phase changes, and can typically go through a large, if not an infinite number of cycles without losing their effectiveness. Organic phase change materials include paraffin waxes, 2,2-dimethyl-n-docosane (C.sub.24H.sub.50), trimyristin, ((C.sub.13H.sub.27COO).sub.3C.sub.3H.sub.3), and 1,3-methyl pentacosane (C.sub.26H.sub.54). Inorganic materials such as hydrated salts including sodium hydrogen phosphate dodecahydrate (Na.sub.2HPO.sub.4.12H.sub.20), sodium sulfate decahydrate (Na.sub.2SO.sub.4.10H.sub.20), ferric chloride hexahydrate (FeCl.sub.3.6H.sub.20), and TH29 (a hydrated salt having a melting temperature of 29.degree. C., available from TEAP Energy of Wangara, Australia) or metallic alloys, such as Ostalloy 117 or UM47 (available from Umicore Electro-Optic Materials) are also contemplated. Exemplary materials are solids at ambient temperature, having melting points between about 30.degree. C. and about 50.degree. C., more for example, between about 35.degree. C. and about 45.degree. C. Also, the exemplary materials have a high specific heat, for example, at least about 1.7, more for example, at least about 1.9, when they are in the state at ambient temperature. In addition, the phase change materials may, for example, have a specific heat of at least about 1.5, more for example, at least about 1.6, when they are in the state at the elevated temperatures.

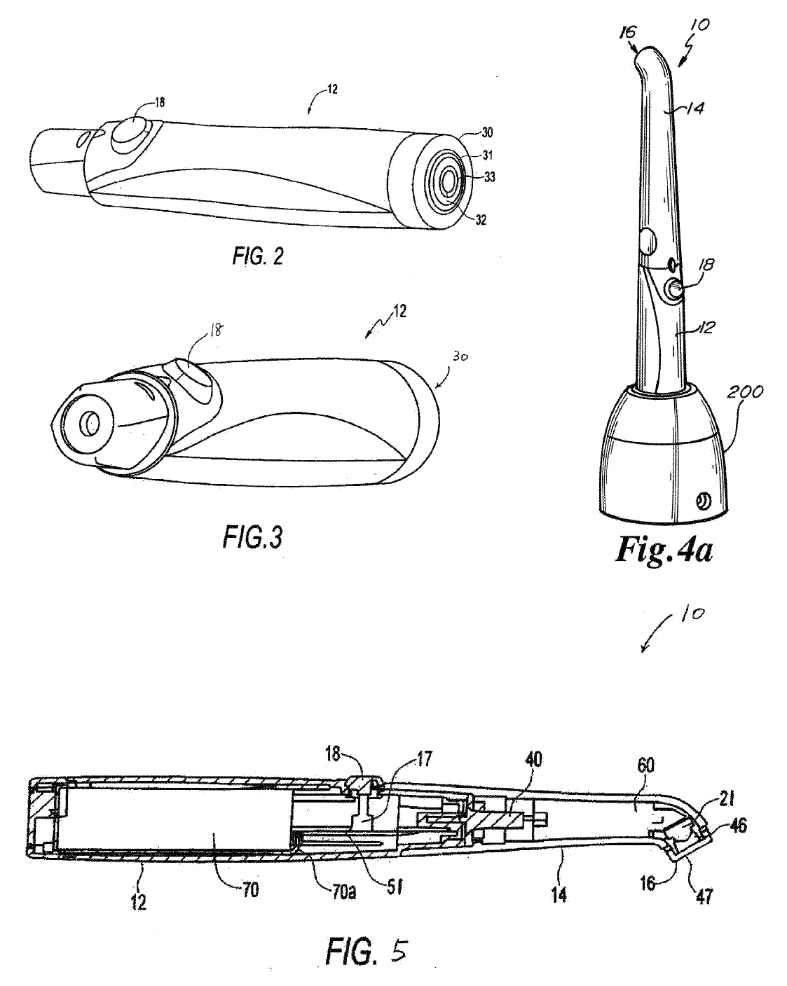

A perspective posterior view and an anterior view of an embodiment of the handle portion 12 are shown in FIGS. 2 and 3, respectively. At the distal end of the handle may be an end cap 30, including, according to one embodiment, electrical contacts 31, 32, 33 so that the curing light may be seated in a charger base (shown in FIGS. 4 and 4a) for recharging the battery 70, if the curing light is battery powered. The end cap 30 and/or the charger base (as exemplified in FIG. 4), may also be so constructed as to provide means for diverting heat away from the curing light after use.

The housing, including its handle portion 12 and front portion 14, may be constructed of a high temperature polymer or composite, such as ULTEM®., which is an amorphous thermoplastic polyetherimide or Xenoy®. resin, which is a composite of polycarbonate and polybutyleneterephthalate or Lexan®. plastic, which is a copolymer of polycarbonate and isophthalate terephthalate resorcinol resin, all available from GE Plastics, or any other suitable resin plastic or composite. At the same time, high impact polystyrene, some polyesters, polyethylene, polyvinyl chloride, and polypropylene may also be suitable.

Polymeric composites, as mentioned above, such as engineering prepregs or composites are also suitable for the composition of the housing. The composites may be filled composites, filled with conductive particles such as metal particles or conductive polymers to aid in the heat dissipation of the device.

An on/off button or switch 18 may be located on the handle portion 12, near the junction between the handle portion 12 and the front portion 14, for manually turning on/off of the curing light. The button may be a molded part, made out of a polymer such as high temperature plastics or polymers used in other parts of the housing, as discussed above. It may also be of the same or different color from the housing. A different color may also help to accentuate its presence and make it easier to find.

It is also worth to mention that the electrical and control components may be received within the housing portions 12 and 14 towards the distal end 13 of the curing light 10. The curing light 10 may be battery powered or tethered to a power source or a transformer. Battery powered curing lights may offer better portability.

Referring to FIG. 1b, a battery 70 may provide electrical power for operating the light source 20 via battery contacts 70a and pin connector 40. In one embodiment, a single rechargeable battery such as a lithium ion battery may be used to power the curing light 10. The on/off button 18 may serve to manually operate the curing light by providing a user input signal through a shaft or post 17, which interfaces with a printed control circuit board 50, may also be located within the handle portion 12, and is mounted close to the battery 70, for example. In one embodiment, a control circuit board 50 includes a device, which may or may not include a microprocessor, which monitors battery life, LED temperature, or system functionality.

The end cap 30 is cylindrical in shape and may be attached to the distal end of the handle portion 12. It may be molded as part of the handle portion 12. It may also be attached by other means, such as adhesive bonding, heat bonding, or threaded attachment.

In one embodiment of the invention, as shown in FIG. 4, the charger base may include an electric motor mechanically coupled to a fan or turbine 201. The fan or turbine 201 may be adapted to draw or urge ambient air across a surface of the heat sink 60 to provide cooling of the heat sink 60. In one embodiment, this cooling may occur when the curing light is at rest or being recharged. In another embodiment, the cooling means is present inside a charger base or cradle 200, for recharging the curing light. In other embodiments, the charger base or cradle 200 may not have a fan 201 or cooling means, but instead or additionally, many include a display panel (not shown) for displaying a condition of the battery. In another embodiment, the heat dissipation device may include a compressed air cooling system.

Still referring to FIG. 4, a separate battery charger module 2200 is included in one embodiment. The charger module 2200 is adapted to receive AC power into a plug 400 from a traditional wall socket and provide DC power to the curing light for battery charging.

The battery charger module 2200 illustrated in FIG. 4 has a cable 428 for conducting electricity from the plug 400 to the charger module 2200. The battery charger module 2200 includes circuitry 430 for controlling battery charging of batteries.

A curing light 10 according to an embodiment of the present invention was used for uniformity measurement.

Method

The Top Hat Factor method for measuring the uniformity of a beam, called the Top Hat Factor, is described below:

An image produced by power falling on one side of a translucent screen is imaged by a video camera. The distance between the camera lens and target are kept constant. The curing light 10 is held rigidly so that its' emitting end or tip 16 is shining directly on the plane of the target being imaged. The target and connected camera are then moved as one unit away from the light curing tip 16 by known distances. At each distance, the image of the beam shining on the target is captured and analyzed. At the zero mm distance (when the tip end 16 is directly against the target screen), the lens aperture is opened so that the camera detector is just below saturation (thus the camera was using the full dynamic range of the capture device). After entering the emitted power level from the light into the software, the computer distributes this power across the detector area and assigned response values for each pixel being illuminated. In so doing, the dispersion of applied power over the target pattern is displayed. The pattern can be seen in 2- or 3-dimensions, using a color graduated scale to indicate the range of power levels present.

The software for beam analysis (LBA-FW-SCOR v4.83.90.11, Spiricon Inc., Logan, Utah) is designed to set a baseline level, so that the response of each pixel is adjusted to be similar. This process is termed “Ultracal!”, and this feature is very important not only for obtaining accurate results of low level signals, but also for the high power ones as well.

The operator enters the total power emitted by the beam into the software. The analytical conditions are also set in software to calibrate the physical dimensions at the target plane, so that accurate length measurements can be made. The levels of cutoff (“clip”) at which the fringe areas are considered not to be a part of the “effective” beam are entered as well. Standard protocol indicates a 20% clip, where peripheral power levels falling less than 20% of the peak power level in the beam are excluded from analysis. The “effective” beam diameter is measured from the power pattern that remains after that clip. In each case tested, the effective beam diameter determined using this technique matched the measured optical diameter of the distal end 16 of the curing light 10.

Once the dynamic range, input power, and effective beam area have been configured, the software then determines the general uniformity of power dispersed across the effective diameter. The method used for this analysis relates to the ability of light to provide a totally uniform level of power at every pixel location. If such a light were analyzed, then the generated figure should appear as a cylinder of specified height (related to the power value), if the beam were circular in shape. The phrase that is often applied to such imagery is a “Top Hat”, where a flat brim surrounds a cylindrical tube (in which the head rests), with a very flat top, as exemplified in FIG. 8. Thus, the relative ability of a light beam to uniformly distribute its power across the effective beam diameter is measured as its Top Hat Factor (THF). A value of unity indicates a beam with perfectly uniform power levels across its top. Values less than one indicate a discrepancy in power distribution, with the lower numbers indicating more discrepancy, and less uniformity.

In one particular example, a curing light 10 according to the present invention having the following arrangement is analyzed using the Top Hat Factor to determine it uniformity. The light source arrangement of the curing light 10 used in this evaluation is shown in cross-sectional view in FIG. 16. The three LEDs 100 were mounted on a substrate 22 which may have a plurality of layers, for example, including a copper layer 221 at the bottom of the substrate 22 to directly contact a mounting surface 61 of a heat sink, (as shown in FIG. 1f and 1k), a fiber glass layer 222 in the middle and a printed circuit layer 223 on the surface on which the LEDs 100 were directly mounted. The copper layer 221 was substantially thicker than the fiber glass layer 222 and the printed circuit layer 223, to more effectively conduct heat generated by the LEDs to the mounting surface 61 of the heat sink 60, as shown in FIG. 1k. A thermister 62 was attached to the outer surface of the heat sink 60 (not shown here) and the arrangement was connected to a connector 80. The thermister wire length X is about 3.5 inches, +/−0.125 inches. The length between the base 22a of the substrate and the distal end 81 of the connector was about 3.625 inches+/−0.125 inches. The dimensions of the substrate 22 is shown in enlarged format in FIG. 16a, including size measurements of the holes formed by side walls 23 and 23′ about the edge of the substrate. The distance between the base 22a of the substrate 22 and the top 22b of the substrate was about 0.4766 inches and the distance between 23 and 23′ was measured to be about 0.52 inches. In this particular arrangement, the LEDs chosen were had a size measurement of about 0.125 inches wide by 0.181 inches long and 0.083 inches tall. They are available from Philips Lumileds Lighting Company San Jose, Calif. with a minimum average output power of 350 mW each when driven by a closed loop constant current control of 950 mA with the 3 LEDs electrically connected in series.

FIG. 16b shows the dimensions of the optical element 46 used in the measurement.

### Beam Homogeneity Measurement

FIG. 11 shows the power distribution of the curing light example described above in mWatts/cm², when the power was accounted for across the bare chips themselves. Very high levels of power density levels were noted in the very center of each chip itself, of up to about 8,500 mW/cm². However, such extremely high readings occur over extremely small surface areas. As seen, there is variation in power output between the bare chips. Also, the power output without the optical element 46 showed hot spots in various locations.

The power density distribution across the emitting end 16 of the exemplified curing light when the optical element 46 was in place and the unit's output was directed at the target is shown in FIG. 12. From these views, it can be seen that there is a fairly uniform power density distribution, until the inner core of approximately 8 mm is reached. There, the triad of LED chips creates a very powerful, isolated area of light, some of which reaches about 1,000 mW/cm² in the immediate vicinity of the chips.

FIG. 13 shows the Top Hat Factor of the exemplary curing light 10 described above. Statistical analysis of these results first looked at how the THF values differed with increasing distance compared to that seen at the emitting end (0 mm) for the exemplary curing light. For this analysis, a one-way ANOVA was performed, using Dunnett's post-hoc unpaired, 2-tailed t-test to compare THF value at 0 mm distance to only that at other tip distances. It can be seen that the power output is fairly uniform with varying emitting end 16 distances from the target. The THF values at 2 and 4 mm distances were not significantly different from that at 0 mm. At distances of 6, 8, and 10 mm, the THF values were lower than that at 0 mm, indicating an increase in beam inhomogeneity with increasing tip distances of greater than 6 mm.

FIG. 14 shows how the beam size increases with distance away from the emitting end 16 for this exemplary curing light 10 of the present invention. As expected, the divergence of power output of this larger beam curing light decreases quite drastically with distance because it generates less power density. FIG. 15 shows the change in power density with increasing emitting end-to-target distance, also for this exemplary curing light. However, for the power output over the entire beam, the exemplary light may actually emit more power at its emitting end than those having a high power density but with a narrower beam diameter.

Having described the invention by the description and illustrations above, it should be understood that these are exemplary of the invention and are not to be considered as limiting. Accordingly, the invention is not to be considered as limited by the foregoing description, but includes any equivalents.

1. A dental curing light comprising:
a substantially cylindrical housing having a distal end, a proximal end having a circumferential span and a housing formation extending more than one-half of the circumference of the proximal end, and a substantially hollow interior;
a light module disposed inside the housing towards its proximal end, said light module comprising a light source, and at least one heat sink for conducting heat away from the light source; and
a lens cap having a peripheral of a span substantially coextensive with the circumferential span of and disposed on the proximal end of the housing for covering the proximal end of the housing;
wherein said lens cap comprises a top surface, a bottom surface and a cap formation between the top and bottom surfaces and extending more than one-half of the peripheral of the lens cap for inter-engaging with the housing formation.

2. The dental curing light of claim 1 wherein said cap formation comprises a peripheral groove.

3. The dental curing light of claim 1 further comprising an optical element positioned proximate the light source.

4. The dental curing light of claim 2 wherein said peripheral groove extends over a center line of the lens cap.

5. The dental curing light of claim 1 wherein said lens cap is detachable from the proximal end when force is applied at certain angles to push the lens cap down.

6. The dental curing light of claim 1 wherein said lens cap and the proximal end of the housing comprises similar material or material having similar coefficient of thermal expansion.

7. The dental curing light of claim 1 wherein said lens cap further comprises a pair of engaging units adapted to correspondingly engage with a pair of receiving slots located on the proximal end of the housing when the lens cap is snapped thereon.

8. The dental curing light of claim 1 wherein said lens cap comprises a convex bottom surface to focus the light emitted from the light source.

9. The dental curing light of claim 1 wherein said optical element comprises a top surface, a plurality of alignment legs extending therefrom, and lenses adapted to direct and focus light emitting from the light source.

10. A dental curing light comprising:
a substantially cylindrical housing having a distal end, a proximal end and a substantially hollow interior;
a light module disposed inside the housing towards its proximal end, said light module comprising a light source, an optical element positioned proximate the light source, and at least one heat sink for conducting heat away from the light source; and
a lens cap disposed towards the proximal end of the housing for covering the proximal end of the housing;
wherein said curing light provides a substantially homogeneous beam at a distance of at least 2 mm away from the proximal end with an effective beam diameter at the proximal end of at least 9 mm.

11. The dental curing light of claim 10 wherein said optical element comprises a light concentrator for focusing light in a direction.

12. The dental curing light of claim 10 wherein said light source comprises an arrangement of three LEDs or groups of three LEDs in a triangle.

13. The dental curing light of claim 10 wherein said optical element includes reflective edges for re-directing light.

14. The dental curing light of claim 12 wherein said optical element comprises bumps for positioning over the LEDs, said bumps having optical properties.

15. The dental curing light of claim 14 wherein said bumps are lenslets covering the LEDs.

16. The dental curing light of claim 14 wherein said optical element comprises a top surface having a plurality of alignment legs extending downwardly therefrom for precise alignment of the optical element with the LEDs.

17. The dental curing light of claim 10 wherein said effective beam diameter is at least about 11 mm.

18. The dental curing light of claim 10 wherein said lens cap has optical properties.

19. A dental curing light comprising:
a substantially cylindrical housing having a distal end, a proximal end and a substantially hollow interior;
a light module disposed inside the housing towards its proximal end, said light module comprising a light source, an optical element positioned proximate the light source, and at least one heat sink for conducting heat away from the light source; and
a lens cap disposed towards the emitting end of the housing for covering the emitting end of the housing;
wherein said light source comprises an arrangement of three LEDs or groups of three LEDs in an equilateral triangle to produce an effective beam diameter at the emitting end of at least 9 mm.

20. The dental curing light of claim 19 wherein said LED arrangement comprises a tight radial configuration or a spaced apart radial configuration.

21. The dental curing light of claim 19 wherein said LEDs are mounted on a substrate comprising formations for aiding the alignment of the optical element.

22. The dental curing light of claim 21 wherein said optical element comprises a top surface having plurality of alignment legs extending downwardly therefrom for matting with the formations of the substrate for precise alignment of the optical element with the LEDs.

23. The dental curing light of claim 19 wherein the light source emits a beam that is substantially homogeneous at a distance of up to about 2 mm from the emitting end.

24. The dental curing light of claim 19 wherein said effective beam diameter is at least about 11 mm.

25. The dental curing light of claim 19 wherein said beam is continuous, pulsed or combinations thereof.
